(19) United States
(12) Patent Application Publication
(10) Pub. No.: US 2005/0240085 A1
(43) Pub. Date: Oct. 27, 2005

(54) Balanced care product customization

(75) Inventors: Rolf Knoell, Laudenbach (DE); Bruno Kaesler, Ludwigshafen (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 11/034,927

(22) Filed: Jan. 14, 2005

Related U.S. Application Data
(60) Provisional application No. 60/600,374, filed on Aug. 11, 2004.
(60) Provisional application No. 60/536,837, filed on Jan. 16, 2004.

(51) Int. Cl. A61B 5/00; A61B 10/00; G06F 17/00

(52) U.S. Cl. 600/300; 128/920; 128/925

(57) ABSTRACT

A method includes obtaining subjective and objective inputs from an individual in need of a custom prepared composition for changing or improving one or more of the individual's conditions. Objective data may be collected by analyzing one or more samples taken from the individual. Subjective data may include data provided in response to a questionnaire by the individual. The objective and subjective inputs are compared against an empirically derived database which links certain affects to variables associated with the subjective and objective inputs.

### BACKGROUND OF THE INVENTION

1. Field of the Invention

The invention relates to a method by which a product is formulated on an individualized basis for an end user by including subjective and objective inputs obtained from the end user and comparing the inputs with an empirically derived database to obtain a balanced formulation. The invention also relates to a method of incorporating the balanced formulation in a production process to rapidly prepare the product. The invention also relates to a method for quantifying subjective inputs from an end user of a product by balancing the quantifiable subjective inputs with objective inputs to prepare a customized product. The invention also relates to a method of preparing or formulating a customized product for an individual by comparing the individual's actual and perceived non-medical condition relative to a scientifically based treatment regimen or against a scientifically derived database of cause and effect principles and relationships. The invention further relates to providing access to higher margin end user (e.g., consumer) markets by a producer of a bulk product by providing individualized preparations to the end user and modifying bulk production processes directly or through a formulator by inclusion of balanced formulation information. The invention further relates to a method of diversifying a customer base to include individual end users by collecting subjective data and objective data from the end users and preparing customized products for the end user.

2. Background Information of the Invention

Individualized and/or customized products are in demand by image and health conscious end users. Individuals with high disposable income who have particularized needs may view products prepared or marketed in bulk or generic form as insufficient for providing a full range of desirable performance characteristics with high efficacy. All commercially available products may be affected by individual preferences. For example, a cosmetic product is a highly personal and individualistic decision on the part of the end user. The cosmetic product's proximity to the individual (e.g., applied directly to the individual) and the potential effects on the end user's appearance are significant.

Many end users of cosmetic products define their identity at least in-part on their appearance. The products which they use to obtain certain image-defining effects and to customize their appearance are often central to an individual's attempt to define identity.

Such end users of cosmetic products have a high quality of life expectation. Quality of life may be dependent on many factors including, body health and personal satisfaction. Body health may include such elements as health and beauty which may be effected by nutritional intake, cosmetics, and pharmaceuticals. Personal satisfaction is a sociological or psychological element which may include feelings of belonging, personal achievement or recognition by others. These elements of personal satisfaction may be exploited commercially through methods including branding.

An increase in the standard of living in many developed countries, a desire for individualized products and the ability to pay for such producers has increased substantially in the last 20 years. A desire for “luxury” products is evident, for example, in the automotive industry where the high-end market has seen continued growth and continued variety. This growth in the high-end is encouraged by manufacturers who benefit from the higher profits associated with such luxury products.

Products that are amenable to individual customization also include durable goods such as automobiles and homes. An individual may customize a home by choosing a particular carpet color or carpet fabric, for example. Likewise an individual may desire to purchase an automobile equipped with options that cast the individual with a desirable image or aura.

Most consumable items may be customized to an individual's personal preferences. Such consumable items include materials which are a part of everyday modern living. Examples are as diverse as dishwashing fluid and motor oil. The degree and effects of customization can range widely. For example, individuals may prefer different degrees of foaming, biodegradability, scent and viscosity in preparations or products that are used to clean household items, e.g., washing-up soaps and liquids. Some consumers may prefer a solid material that dissolves quickly and provides high foam whereas other consumers may prefer a liquid preparation that disperses slowly yet provides the user with a pleasant scent after use. The specific performance characteristics of a product may be tailored through changes in the product's chemical formulation, such as, for example, the types and/or amount of dispersant and/or counter-ion present in a detergent. Many consumers prefer certain colors or scents which may not directly impact the ability of the material to carry out its desired function but instead impart aesthetic preferences to the material.

In many markets that are directly or peripherally affected by consumer preference for a customized product, (e.g., nutriceuticals, pharmaceuticals and cosmetics) the number of product variants offered in many particular product classes has increased dramatically in the last 20 years. This is evidenced by, for example, the number and types of different shampoos available from any given manufacturer. In today's market, most manufacturers provide varieties of shampoos marketed for niches such as dry scalp, oily scalp, fine hair, thick hair, damaged hair (split ends), dandruff, etc. Many consumers are however overwhelmed by the variety of choices and typically decide which product to purchase through a trial and error process.

There is no restriction on the types of materials that may form the basis of a custom preparation or product. Most non-durable and consumable items may be produced for sale on a custom basis. While liquids may be especially amenable to such customization because they are easy to mix and blend, solid materials such as consumable household chemicals may also be customized. Further, consumable items such as toilet paper may also be customized through texture, scent, and pattern.

Product customization may be critical in some applications such as medicine and pharmaceuticals. A doctor may individually specify the exact dosage and type of a medicine dispensed to an individual to cure a disease (i.e., through a prescription). In most cases this level of customization is limited to the dosage and the selection of a particular pharmaceutical agent from a broad range of commercially available pharmaceutical agents.

Customization may also occur at the end user level when the end user initiates the purchase of a desired product. During the purchase or sales process, a representative from the selling organization (who may in some cases be a consultant or a third party who is not necessarily directly employed by the organization that produces or is responsible for the sales of the product) may provide individual advice and guidance to the individual purchasing the customized product. Such sales methods are often carried out in home improvement stores when an individual selects a specially blended paint color. Factors which may affect the characteristics of the paint (such as gloss, color, shade, cost, cleanability, quality, etc.) are specified by the customer and communicated to the representative from the selling organization. The sales representative may then provide additional advice and know-how to allow the individual to make a better informed purchase decision. Today, in many cases, the individual human sales representative has been replaced with an automated system which may use a computer to guide an individual through the product selection process and automatically customize a product based upon the specific data inputted into the computer by the end user.

Cosmetic and nutritional preparations, sometimes referred to as neutraceuticals, are areas where a high degree of customization is often needed in order to satisfy the end user's demands. Customization in the cosmetics industry is described for example in U.S. Pat. No. 5,622,692 to Rigg et al. Rigg discloses a method that allows an end user to input variables including skin tone into a computer apparatus to develop a customized cosmetic formulation. The method of Rigg may include an objective measurement of the end user's natural skin coloration. The skin coloration data is obtained by measuring the end user's skin color at the point of sale with an electronic device. The natural skin coloration input is captured by the computer apparatus and is compared with the end user's desired skin color, skin shade, skin oiliness, etc., properties which are also inputted into the computer by the end user. The Rigg method therefore includes an objective measurement of skin color with a subjective measurement inputted by the end user to prepare a cosmetic which is mixed, dispensed and provided to the customer at the point of sale. A sales agent or consultant may be present during the measurement of skin coloration or during the inputting of the end user's subjective inputs to provide advice and guidance at the point of sale.

The subjective input is important because it reflects the end user's desired performance characteristics. The consultant or sales representative interfacing with the end user may help the end user make subjective judgments based upon the consultant's experience, training and/or preferences. An end user describing subjective inputs to a first consultant may obtain a product that is substantially different from a product obtained by interfacing with a second consultant although the end user has provided the same subjective inputs. There may therefore be a lack of consistency encountered when interpreting the subjective inputs provided by the end user.

An end user purchasing a customized product for nutritional or dietary needs, such as a neutraceutical, may be especially sensitive to the performance and effects of the product. Inputs based only upon measurements taken at the point of sale together with subjective inputs included with or without the guidance of a third party or consultant may not be sufficient for providing a custom prepared product that is fully responsive to the end user's requirements and demands. Inputs in such cases, when obtained from an on-site instrument or analytic technique, may ignore important aspects of the end user's health condition which may adversely affect the performance of the custom prepared product.

Face creams customized to an individual end user based upon a DNA test are currently available. The end user provides a DNA sample to a third party or alternatively self-administers a DNA test and provides further information through a profiler that provides data on the end user's skin condition such as oiliness, texture, pore size, and skin condition. Subjective inputs such as the environmental conditions around the end user are included in the profile. The skin profile provides information with regards to the end user's inputted data, such as whether or not the end user is a smoker, and provides data with regards to the effect of that activity upon the skin condition of the end user. The end user may input the subjective data through a computer network located either at the point of sale or in the end user's home.

Conventional methods of customizing a product based on inputs received from the end user, whereby an individual provides a tissue or body fluid sample for analysis and a custom preparation is subsequently prepared, lack the guidance of a technically trained consultant or store front manager and further lack the opportunity for feedback from the end user which may otherwise be used to optimize the formulation for the end user. It is therefore difficult to improve such systems through an improvement process that relies on an enriched database.

Several methods where the end user intensively interacts with another party to develop a treatment regiment are known and include such traditional healing methods including holistic treatment.

Holistic and whole body treatments are ways of healing the body by using techniques and treatments drawn from ancient and contemporary culture. These techniques are sometimes referred to as integrated health therapy or complementary health therapy because they may involve techniques from several disciplines including medicine, fitness, cosmetics as well as other conventional techniques.

No formal training is required to practice holistic healing which has a limited number of standardized regimens. Holistic practitioners may take their influence from a wide variety of sources. Holistic healing does not require the discipline of the scientific process and may actually place an individual in worse condition by treating only those symptoms which an individual wishes to address while ignoring other potentially life threatening illnesses or imbalances of the body.

Traditional methods that may be used in holistic healing include for example acupuncture, aromatherapy, ayurvedic herbs, biofeedback, bodywork, cell therapy, chelation therapy, chiropractic, colon therapy, detoxification therapy, energy medicine, flower remedies, guided imagery, herbal medicine, holistic referrals, hydrotherapy, hypnotherapy, ionized air (negative ions), light therapy, meditation, mind/body medicine, naturopathic medicine, neurolinguistic programming, orthomolecular medicine, oxygen therapy, testing for stomach acidity, traditional chinese medicine herbs, and yoga.

Holistic practitioners are often viewed as teachers who educate, empower, and motivate their patients or clients to lead healthier lives, emphasizing prevention as the best cure. Holistic medicine may be used in conjunction with conventional medicine. Typically, holistic practice does not base a treatment regimen on scientific principles applied by a technically trained and non-medical practitioner.

Holistic healing is viewed by many as an “alternative” or “complementary” medicine and is a controversial although sometimes effective means of treating illness. In part due to this controversy and other criticisms, holistic healing has not gained wide spread acceptance among the broader population that is the client base for the medical community. Traditional health and cosmetic practices lack the guidance of a practitioner who is technically trained and do not provide an objective referral and feedback system.

Homeopathic medicine is the practice of using extremely small doses of medicines and herbs to cause the same symptoms the disease causes. Homeopaths (practitioners of homeopathy) acknowledge no diseases, only symptoms.

The traditional sales process for providing customized product solutions benefits the end user by allowing the end user to find or formulate a product that exhibits particular performance requirements and characteristics. Since the inclusion of the end user's inputs into the product selection process occurs during a face-to-face meeting between the sales representative and the end user (or in the case of an automated system at the point of sale) the premium paid by the end user to have customized features included in the product may be captured at the point of sale or at any point of contact to which the end user provides the desired product specifications (inputs). The producer of the raw materials necessary to produce or prepare the customized product normally may not share in the price premium paid by the end user.

The profit margin associated with producing a bulk, generic or semi-commodity material such as a paint or cosmetic may be significantly lower than the margin at resale (e.g., a single digit profit margin vs. a double digit profit margin). Access and/or direct sales to the end user would be desirable for the producer of the bulk materials because the bulk producer may then be able to capture at least a portion of the profit margin which is normally captured at the point of sale. It is difficult however for the bulk producer to participate at the point of sale because a bulk producer normally does not have the sales expertise, distribution chain, organization or know-how to reach horizontally through the product chain from production of the raw materials to the point of sale. The business organization of a bulk producer may also be subject to inefficiencies due to differences in management techniques and organizational priorities that may be present in a production organization in comparison to a sales and/or consulting organization.

A method by which a bulk producer may access individual end users to prepare individualized preparations or products based upon the end user's desired performance specifications may provide a way to realize significant efficiencies in production and distribution costs by eliminating entire levels of intermediaries who may otherwise separately expect an economic return for their participation in the product chain.

Therefore, methods exist by which intensive one-on-one treatment or consulting is available with a knowledgeable party. Such consultations however are limited by the lack of a repeatable process by which knowledge and information is gathered to continually improve the quality of the consultations or diagnosis. Where traditional methods may provide a customized process for an individual after a long consultation period, such learning is difficult to impart to different end users and is based in some cases entirely on a subjective process. Customized products that are developed specifically for end users based upon a combination of the end user's subjective and objective inputs may provide a way of quickly and reliably identifying a product for sale to the end user exhibiting the desirable performance characteristics.

WO 2004/091501 A2 discloses a process of determining individual skin structure and function at a point in time for the purpose of determining and formulating skin care products that remedy the deficiencies observed in the skin. The process comprises the steps of obtaining at least one biometric measurement on an individual's skin to form a data set and of creating the skin care product for the individual based on this data set. The process further comprises the step of the individual filling out a lifestyle questionnaire

### SUMMARY OF THE INVENTION

The invention includes a method of preparing a customized product responsive to objective input (criteria) obtained through analytical measurement of an end user, or of a sample provided by the end user, and subjective input (criteria) obtained from the end user, for example by a questionnaire, to provide a customized, high value product for the end user. The subjective input may include data regarding the performance criteria (e.g., functionality) desired by the end user in the customized formation. The subjective and objective inputs may be cross-correlated by comparing the inputs to a scientifically derived and organized descriptive overview (e.g., a database) of how one or more known components of a formulation responds to the inputs.

In another embodiment of the invention, a manufacturer or provider of a raw material, generic or bulk product is able to access high margin end user markets by providing custom prepared products for specific end users. The manufacturer includes inputs provided by the end user including subjective and objective data to modify a production process and thereby prepare a custom lot or sublot of material for the end user. The method allows the bulk producer to circumvent one or more intermediate levels of distribution, blending, packaging or wholesale and thereby realize an improved profit margin on what may otherwise be sold as a bulk commodity product.

The invention also covers a computer program with program code which is suitable for carrying out a process according to the invention as described above when the computer program is run on a computer. The computer program itself as well as stored on a computer-readable medium is claimed.

It will be understood that the features mentioned above and those described hereinafter can be used not only in the combination specified but also in other combinations or on their own, without departing from the scope of the present invention.

The invention is schematically illustrated in the drawings by means of an embodiment by way of example and is hereinafter explained in detail with reference to the drawings. It is understood that the description is in no way limiting of the scope of the present invention and is merely an illustration of a preferred embodiment of the invention.

### BRIEF DESCRIPTION OF THE DRAWINGS

FIG. 1 shows a diagram of the inclusion of objective and subjective data from an end user to a formulator for producing a product for delivery to the end user.

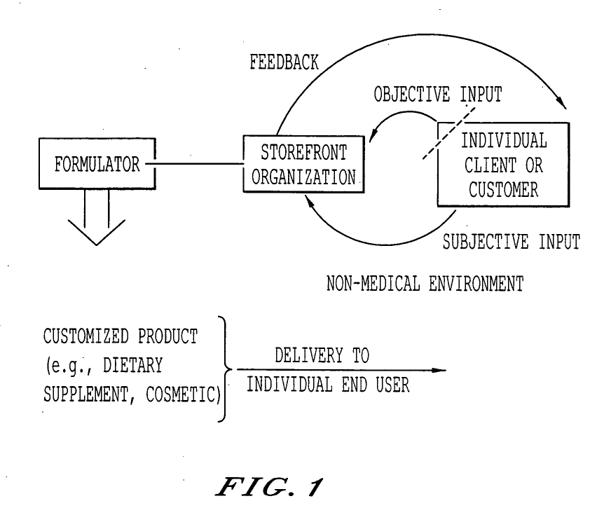

FIG. 2 shows a diagram of a computer system which may take inputs from an end user through a client or a point of sale data entry terminal.

FIG. 3 shows a client/server structure which allows data (inputs) to be transferred form an objective database and/or subject database, one or more of which may be stored in one or more servers or a duplicate thereof may be stored on a client, and transfer of the data over a computer based network such as the Internet.

FIG. 4 is a diagram showing communication flows and product flows and interactions between front-end and back-office functions.

FIG. 5 is a diagram showing a central IT system structure for an integrated implementation of a balance care product customization.

FIG. 6 is a diagram illustrating one embodiment of a process to derive a formulation for a customized product.

FIG. 7 is a diagram illustrating another embodiment of a process to derive a formulation for a customized product.

### DETAILED DESCRIPTION OF THE PREFERRED EMBODIMENTS

In one aspect of the invention process, subjective and objective inputs obtained from an end user are compared to an empirically derived database to obtain customized products for the end user. In this aspect of the invention the subjective inputs identified by the end user are matched with actual test results representing one or more objective inputs. The balance of subjective and objective inputs provides a template against which a customized product formulation can be derived. The template which contains the objective and subjective inputs is correlated against an empirically derived database. The correlation allows an automated selection process to be undertaken whereby certain variables are identified for further action. Once a variable, such as, for example, a nutrient level in the bloodstream of the end user, is identified and matched with the end user's subjective and objective inputs, a composition can be formulated that may address variables which may exhibit sensitivity to either the symptoms or condition with which the end user is afflicted or which the end user otherwise wishes to modify.

An objective referral system is one in which the treatment data for many individuals is saved. The database becomes enriched as more data from an increasing number of individuals is included. This then allows the application of scientific principles by which the data can be analyzed and the accuracy of customization improved.

The information derived from the objective and subjective inputs is then included in the formulation of a specific product for the end user through a preparation process undertaken at a facility that may be remote from the point of sale or remote from the point at which the end user submitted the subjective and/or objective inputs. Preparation of the product may take place at a third party or at an in-house preparation operation. During preparation one or more ingredients available in bulk form are mixed or blended. The customized products are prepared by varying the relative ratios of each of the components of the preparation, by varying the selection of components present, and/or by varying the form of the formulation. The preparation may be prepared in any physical form. Preparations as individual doses such as pills, powders, pastes, oils, waxes are included.

After the subjective and objective data has been entered into a computer and cross-correlated to the empirically derived database it is analyzed and a custom, specific formulation is generated. The formulation is then converted into electronic signals which may be transmitted to an automated formulator or other device or apparatus located remotely which produces a preparation for an end user in the desired quantity with the desired properties.

Once prepared the product may be shipped directly to the end user or alternatively may be distributed to the point of sale where the end user may make payment and finalize receipt of the finished product.

The end user may initiate the process by visiting a point of sale or be visited by a representative for the bulk producer/formulator or a combination thereof. For example, an individual may desire a skin lotion designed specifically for the individual's skin condition. The lotion may contain, for example, emollients, water, oil, and/or personal care agents. An objective input into the invention process would include an analysis of the individual's condition that may include the degree of oiliness of the skin, texture and other indicators that may be reflective of skin health condition. The objective and subjective inputs may help to determine the dosage and identity of specific ingredients such as the active agents and the relative ratios of ingredients including oils and emollients.

The subjective data inputted by the end user is matched with the objective inputs to develop the final formulation. The subjective input may be obtained by, for example, deriving lifestyle information and/or the desired performance characteristics from the end user. The subjective data may be obtained, for example, at the point of sale, by consultation with a third party such as a technically trained person, or by completing a questionnaire accessed over an electronic network through a published document. In another embodiment, consultation is carried out in the presence of a pharmacist, a doctor or other professional.

The customized product may be any kind of cosmetic or nutritional preparation but also any other kind of preparation for internal or external application by an end user suitable for customization.

For a formulation such as a skin cream the subjective data may include the desired appearance of skin and/or the desired skin tone and/or the perceived performance attributes of the product provided to the end user. Other subjective data may include the degree of exposure to sun, prior cosmetic use, smoking habits, exercise/diet, sleep patterns, allergies, posture and others. In the case of a formulation for an extra nutrition product (dietary or nutritional supplement or neutraceuticals), the subjective data may also include eating/dietary, drinking and smoking habits, exercising habits, prescription situation (such as hormone and non-hormone prescriptions), medical condition and medical (family) history, environmental exposure, allergies etc. The subjective data is collected in an electronic form and matched with the objective inputs. The cross-correlating process produces a balanced formulation that takes into account both the objective input (i.e., that data which is objectively reflective of the end user's health condition and may be obtained through blind analytical techniques) and subjective input (i.e., data that may be reflective of the end user's desired characteristics and personal impressions on the end user's condition). The formulation is thereby balanced to take into account both the health condition and the subjective desires of the end user.

Once a formulation has been derived it may be used in a manufacturing process to produce a predetermined quantity of a custom product in a predetermined package type for the end user. Production of the product provided to the end user may be carried out in an automated system that is networked to the system which may store and transfer the balanced care formulation to the automated process. Upon receipt of a confirmed order from the end user, a signal and data packet may be sent to the production area. In the case of, for example, a skin lotion, the information may be sent to a preparation line which is capable of dosing the desired amounts of individual ingredients defined by the balanced care formulation into an individual package size. The preparation thus prepared and packaged is identified in a manner that uniquely identifies the end user. In a further automated process the individual package of the produced product is prepared for shipment or distribution to the end user. The package may be delivered directly to the end user or first to the point of sale for pickup by the end user.

The producer of one or more of the ingredients present in the preparation prepared for the end user may have ownership or responsibility for oversight of one or all of the processes described herein. Therefore, a producer of, for example a skin oil may have a storefront employee located at a site remotely from the production facility. The remote location of the storefront employee may be desirable because it allows direct face-to-face interaction with the end user. The process allows the producer of one or more of the bulk ingredients used in the preparation to have direct access to the end user. Direct access to the end user allows the manufacturer access to a high value segment of the business chain. In one embodiment the manufacturer may be viewed as being horizontally integrated from the point of manufacture of one or more of the base ingredients of the end user's desired product to the sale and consultation with the end user.

By accessing the higher value end market a manufacturer of bulk goods may significantly improve the profit margin attainable from what is normally a low price or low margin commodity good.

In preparing a balanced care formulation one or more of the following actions may be undertaken together with any number of additional actions. A first step may include analysis of the end user's medical condition (e.g., collecting the objective inputs). Analysis may include, for example in cosmetics; moisture (hydration), sebum (oil), skin pH, skin elasticity, skin color, skin pigmentation, skin barrier function (TEWL), skin temperature and skin surface structure. The analytical measurements may be linked or correlated to components of the formulation that may exhibit one or more specific physiological and/or dermatological effects. Such measurements may include genetic testing to help determine the traits of the end user. Many methods are conventionally known for measuring these parameters. In dietary supplements a broad range of substances can be determined in a broad range of body materials including blood, serum, urine, stool, sweat, saliva, buccal mucosa cells, and hair for substances such as vitamins (A, B₁, B₂, B₆, B₁₂, C, D, E, K, Folic acid), minerals (Ca, Mg, Fe, Cu, Zn, Se, I), and others such as β-carotin, as well as diabetes, elevated blood lipids, endometriosis, gallbladder, fibromyalgia, fructose intolerance, lactose intolerance, psoriasis etc. but also general health analysis regarding sicknesses and their healing, kidney stones, chest pains, headaches and migranes, cramps etc.

Analysis of the end user and balancing of the subjective inputs with the objective inputs is preferably carried out after analysis. The diagnosis of the end user's analytical results (e.g., objective inputs) may include a medical analysis. It may be preferred to communicate the objective data to the end user before product preparation or consulting to modify the subjective inputs from the end user. The communication of the diagnostic results may significantly effect an end user's perception of what performance characteristics may be desired in the product. Communication of the analytical results and diagnosis to the end user may be done in a consulting atmosphere at the point of sale or through a network connection directly to an individual in a consulting capacity or through an automated system. Consulting is an opportunity to modify the subjective inputs from the end user for inclusion in the final product. For example, a pale skinned person wishing olive skin may be counseled that freckles may result upon excess artificial skin coloring. Consultation may also be undertaken in conjunction with sales to establish a longer term supply agreement with the end user.

The objective and subjective data is compared against a database of scientific data which may contain data developed empirically to determine the essential and most beneficial ingredients in the customized formulation. Such scientific data may come from many sources such as treatises of the human condition, health encyclopedias, data derived from ancient texts, anecdotal evidence, or scientifically appended performance observations derived from one or more end user's prior customized formulations or preparations. The database is preferably in an indexed electronic form to provide for quick and reproducible analysis. One or more algorithms may be used to balance or compare the inputs with the database. The formulations may also be tested on a theoretical basis against a model performance algorithm which may help to indicate and identify the timing of particular milestones or physiological changes that may accompany the administration or application of the specialized preparation.

The term “treatment” as used herein refers to the administration of a substance or a composition to a person in any manner (e.g., orally, topically, subcutaneously) and does not require the administration of a prescribed medicine or other medically initiated activity as a response to a medically diagnosed disease or condition.

The term “objective data” in the context of this invention comprises analytical physiological test values and parameters.

The term “subjective data” in the context of this invention comprises non-analytical data related to the body constitution of the end user, and may also include so-called lifestyle information.

The term “customized product” includes blends, mixtures and package types prepared for a specific user responsive to objective and subjective criteria analysis.

The term “formulation” as used herein refers to a composition or a recipe on the basis of which a customized product is prepared.

The term “scientific method” is used herein to refer to a process of balancing different variables by incrementally changing variables to arrive at an optimum solution.

In one embodiment of the invention inputs from an end user are collected and used to modify a production process to prepare a custom prepared product for the end user. The inputs from the end user include objective and subjective inputs. The process may be a large scale industrial-type process which produces, for example, from 10 to 10,000 pounds of material per hour. Alternatively the production process may be a traditional pharmacist type mixing process wherein small quantities for individual doses (about 5 mg) to multiple doses of different materials are individually mixed, blended and packaged etc. to meet the specific short term needs of a customer.

Objective inputs include, for example, analytical measurements which may be carried out on tissue samples or by other medical, physiological or dermatological analyses of the end user. Analysis may include analysis of the skin for oiliness, texture, coloration or other properties of the skin. Other tests may include analysis of bodily fluids such as urine, blood or sweat and may be carried out in order to determine for example electrolyte level, and other aspects of the human condition.

Subjective inputs may include a wide range of performance properties that may be desirable in a customized product prepared for a specific end user. The end user may, for example, desire a product which provides high luster to the hair. A different end user may prefer a product which provides low gloss but high body. Other subjective inputs may include lifestyle choices of the end user. Such lifestyle choices may include, for example, the weather environment in which the end user is normally present, the degree of physical exertion experienced by the end user on a day-to-day basis, exposure to the sun or smoke, tobacco consumption and others.

The objective and subjective inputs taken from the end user may be balanced with an empirically derived database in order to provide the formulation desired by the end user. For example, an end user may request a shampoo or hair care product that exhibits characteristics including low impact upon the scalp, body for thin hair, and low luster. Subjective data may then be combined and correlated with analytical measurements of the end user's tissue which show that the end user has, for example, oily skin. Comparison with the empirically derived database provides a way to validate the end user's subjective inputs while concurrently balancing the formula so that no unintended affects arise. While a balanced determination may not appear to be critical to the choice of a hair care product, a special formulation may counterbalance oily scalp with brittle hair, for example. Identification of the underlying condition may allow formulation of a product which is able to balance apparently countervailing effects such as skin oiliness on the head and skin dryness on the hands to provide an overall neutral, non-dehydrating and non-sebum activating preparation. The subjective data may then be included in the formulation by adding or balancing specialty additives or other materials. The end product is prepared specifically to provide the desired performance characteristics of the end user.

In dietary supplements, the invention, based on a subjective and objective analysis of the health and dietary situation of the end user, provides for a nutrional supplement product which enables the end user to obtain a higher level of a balanced diet with view to his or her specific lifestyle. Consulting the end user upon receipt of the result of the analysis may help him or her to change the lifestyle situation. A change in lifestyle may be determined during a subsequent test and consultation when the end user is seeking an updated customized dietary supplement product.

The formulation of a customized product for an end user is not necessarily symptom driven. In contrast to homeopathic medicines which may be solely symptom driven, a customized product based upon an individual's balanced subjective and objective inputs may be used in a prophylactic manner rather than in a manner which requires the administration of the preparation to address only immediate and/or apparent health problems (e.g., symptoms). An individual may for example seek guidance on obtaining a preparation which provides good hair treatment and at the same time seek advice on the preparation of the same or a different product which may reduce excess flatulence. Regardless that medical terms may exist for symptoms such as dry hair or excess flatulence, numerous over the counter products are commonly available to treat these symptoms in the balancing formulations for most individuals. The submission of the subjective and objective inputs and comparison of the data (i.e., inputs) against an empirically derived database linking variables, performance characteristics and physiological effects by a technically trained consultant may provide an optimum solution for the end user without requiring the intervention of a medical doctor or the application or administration of regulated materials.

The empirically derived database may include data already broadly known and widely accepted in the medical community. The empirical database may contain a matrix of essential nutrients for humans and may correlate the identity of a nutrient with a dosage range and may further correlate the effects of the nutrient on health or other aspects of the human condition when the nutrient is not present in the desired amount. Examples of such nutrients include vitamins, vitaminoids, minerals, trace elements, amino acids, phytocompounds, antioxidants, algaes, and coenzymes. Examples of such compounds and typical dosage ranges are tabulated below.

Vitamins/Vitaminoids (Antioxidants)

| Compound | Dosage-Range | |
|---|---|---|
| L-Ascorbic Acid (Vitamin C) | 200-1,000 | mg |
| Bioflavonoids (Aserola-, Citrus-) | 200-250 | mg |
| Ascorbylpalmitat (lipid-soluble Vitamin C) | 20-50 | mg |
| d-Alpha-Tocopherol (Vitamin E) | 50-400 | mg |
| Mixed alpha-, beta-, gamma- delta-Tocopherols/ | 50-200 | mg |
| Tocotrienols | | |
| Mixed Carotinoids (e.g., Betatene ™) | 10 | mg |
| Lycopene (e.g., Lyco-O-Mato ™) | 2-4 | mg |
| Zeaxanthin | 2.5-5 | mg |
| Lutein | 5-10 | mg |
| Vitamin A (Palmitate) | 2,500-5,000 | IU |
| Cholecalcifertol (Vitamin D) | 200-400 | IU |
| Vitamin K1/K2 | 2-5 mg/0.5-1 mg | |
| Alpha Lipoic-Acid (ALA) | 100-400 | mg |
| Ubiquinol (Coenzyme Q10) | 30-200 | mg |
| Oligomer Proanthocyanides, OPC (e.g., Activin ™) | 25-50 | mg |

B-Vitamins

| Compound | Dosage-Range | |
|---|---|---|
| Thiamin, Vitamin B₁ | 10-50 | mg |
| Riboflavin, Vitamin B₂ (incl. RBFL-5P 2 mg) | 10-50 | mg |
| Niacin, Vitamin B₃ | 10-50 | mg |
| Panthotenic acid, Vitamin B₅ | 100-200 | mg |
| Pyridoxin, Vitamin B₆ | 10-50 | mg |
| Pyridoxalphosphat, activated Vitamin B₆ | 1-4 | mg |
| Cyanocobalamin, Vitamin B₁₂ | 400-1,000 | μg |
| Methylcobalamin; Vitamin B₁₂ | 100-1,000 | μg |
| Folic acid | 400-800 | μg |
| Biotin | 500-1,000 | μg |
| Trimethylglycine, Betain | 150-250 | mg |

Amino Acids

| Compound | Dosage-Range |
|---|---|
| L-Proline | 250-500 mg |
| L-Tyrosine | 250-500 mg |
| L-Lysine | 250-500 mg |
| L-Carnitine | 250-500 mg |
| N-Acetyl-L-Cysteine | 250-500 mg |
| Taurine | 250-500 mg |

“Good Lipids”

| Compound | Dosage-Range | |
|---|---|---|
| Eicosapentaenoic acid (EPA) | 500-750 | mg |
| Docosahexaenoic acid (DHA) | 300-500 | mg |
| Alpha-Linolenic acid (e.g., from Perilla oil) | 1,000-2,000 | mg |
| Gamma-Linolenieic acid (e.g., from Borage) | 100-200 | mg |
| Cholina | 50-00 | mg |
| Phosphatidylchloline | 100-150 | mg |
| Inositol | 100-200 | mg |
| Soy lecithin (99% oil free, 97% phosphatides) | 500-1,000 | mg |

Trace Elements

| Compound | Dosage-Range | |
|---|---|---|
| Zinc (Opitzinc ™) | 10-30 | mg* |
| Selan (from yeast, or an organic as sodium selanite) | 50-200 | g* |
| Boron | 1-3 | mg* |
| Calcium | 200-300 | mg |
| Magnesium (e.g., magnesium-aspartate, magnesium | 150-50 | mg* |
| oxide) | | |
| Chromium (e.g., chromium polynicotinate) | 100-200 | μg* |
| Molybdenum (e.g., sodium molybdate) | 50-00 | μg* |
| Vanadium (e.g., sodium methavanadate) | 5-10 | μg* |

*elemental yield, no manganese, iodine possibly

Phyto-Compounds (Vegetable-Fruit Complex):

| Compound | Dosage-Range | |
|---|---|---|
| Resveratrol (Regrape X ™) | 4-5 mg | |
| | (50-200 mg) | |
| Indole-3-Carbinol (13C) | 100-200 | mg |
| Bioflavonoids (Acerola-, Citrus-) | 200-250 | mg |
| Quercetin | 50-300 | mg |
| Pycnogenol ® | 10-25 | mg |
| Saw palmetto CO2 extract | 150-300 | mg |
| Nettle-leaf extract | 500-1,000 | mg |
| Bilberry | 25-100 | mg |
| Green-Tea Extract (95% polyphenols, decaff.) | 100-200 | mg |
| Grapeseed Extract (Leucoselect ®, BioVin ® or | 10-25 | mg |
| Masquelier's ™) | | |
| Oligomer Proanthocyanidinines (OPC, e.g., Activin ™) | 25-50 | mg |
| Milk thistle extract (85% silymarin) | 50-100 | mg |
| Raspberry extract (38.5% ellagic acid) | 50-120 | mg |
| Acerola juice powder | 100-300 | mg |
| Broccoli concentrate | 250-500 | mg |
| Ginkgo biloba extract (24% ginkgo flavones) | 50-120 | mg |
| Curcumin (76.1%, e.g., from turmeric root extract) | 500-650 | mg |
| Bioperine ® from black pepper | 2.5-5 | mg |
| Garlic extract (e.g., Kyolic) | 750-1,000 | mg |
| Soy-extract (40% isoflavone extract) | 150-300 | mg |
| Spirulina (blue-green algae) | 100-200 | mg |

Mitochondria-Related Compounds

| Compound | Dosage-Range | |
|---|---|---|
| Acetyl-L-carnitine | 1,000-2,000 | mg |
| Creatine | 1,000-3,000 | mg |
| Alpha Lipoic-Acid (ALA) | 100-400 | mg |
| Ubiguinol Coenzyme Q10 | 30-200 | mg |
| Oligomer Proanthocyanides, OPC (e.g., Activin ™) | 25-50 | mg |
| Carnosine | 500-1,000 | mg |

The empirically derived database may further include any data that is derived by scientific principles through study of the human condition. Such empirical data is not limited to medical conditions but can also encompass the physical effects determined by the scientific method upon, for example, hair, skin, keratin, by testing of various compounds and determining the effect of the compound on the hair, skin etc.

The effects of any specific compound may be correlated in the database through links to the subjective and objective inputs obtained from the end user. Data obtained from an end user questionnaire regarding, for example age and gender may affect the desired dosage range of one or more nutrients or other ingredients. This may be correlated with objective data such as the concentration of the nutrient obtained from, for example, a blood sample. This is then further correlated with subjective data provided by the end user such as, for example, the end user's desire to obtain deeper sleep or have higher energy during the day. An example of the correlation of different nutrient compounds with the links to the end user's subjective and objective data together with data taken from an end user questionnaire is provided in the table below.

In one embodiment of the invention method it is not necessary that the balancing of the subjective and objective inputs and/or identification of an ultimate product or formulation be made by a medically trained individual. In a preferred embodiment of the invention, the person communicating with an individual end user who is considering the purchase of a customized product is not a medical doctor. The consultant or storefront operator in contact with the individual is however technically trained. Technical training insures that the scientific method is utilized when developing a formulation for an end user. Scientific training permits the consultant to operate with a degree of knowledge and on a technical level that is otherwise not attainable by practitioners in, for example, holistic healing. On the other hand, since the consultant is not a medical doctor the consultant is not in a position to recommend treatment for a medical condition. Therefore the service provided to the end user may be limited to those applications that include unregulated cosmetics and dietary supplements with advice and consultation limited to those materials known for their health-promoting effects.

VITAMINS/VITAMINOIDS WITH ANTIOXIDANT PROPERTIES

| | Priority- | Dosage-Range | RDA/AJ | UL | | |
|---|---|---|---|---|---|---|
| Compound | Level | [% of UL] | *[mg] | [mg] | Links to customer data proof | Lit |
| L-Ascorbic Acid (Vitamin C) | 1 | 200-1,000 mg | 90 | 2000 | *Questionnaire: Age, Gender, Problems with | |
| | | [10-50%] | | | mixed connective tissue (MCT), bruising, | |
| | | | | | respiratory infections, season, | |
| | | | | | *Serum: 1 ml [5-15 μg/ml,], transport frozen | |
| | | | | | *SNP; COLIA1, Apo E, ACE, | |
| | | | | | *Technical device: Osteoson®-Kollagenoson, | |
| | | | | | Revisoometer RV 600, Osteodin, | |
| Bioflavonoids (Acerola-, | 1 | 200-250 mg | — | — | *Questionnaire: Problems with mixed connective | |
| Citrus-) | | | | | tissue (MCT), bruising, respiratory infections, | |
| | | | | | winter . . . | |
| | | | | | *Serum:- | |
| | | | | | *Technical device:- | |
| d-Alpha-tocopherol-Succinat | 1 | 50-400 mg | 15 | 1000 | *Questionnaire: Conditions with accompanying | |
| (Vitamin E) | | [5-40%] | | | malabsorption of fats; e.g., gallbladder | |
| | | | | | and pancreas insufficiency. | |
| | | | | | *Serum: 5-20 μg/ml | |
| | | | | | *SNP: . . . | |
| | | | | | *Technical device:- | |
| Alpha-tocotrienol | 1 | 7.5-10 mg | — | — | *Questionnaire: Conditions with accompanying | |
| | | | | | malabsorption of fats, e.g., gallbladder and | |
| | | | | | pancreas insufficiency. | |
| | | | | | *Serum:- | |
| | | | | | *Technical device:- | |
| Beta-tocopherol | 1 | 1-2 mg | — | — | *Questionnaire: Conditions with accompanying | |
| | | | | | malabsorption of fats, e.g., gallbladder and | |
| | | | | | pancreas insufficiency. | |
| | | | | | *Serum:- | |
| | | | | | *Technical device:- | |
| Gamma-tocopherol | 1 | 50-100 mg | — | — | *Questionnaire: Conditions with accompanying | |
| | | | | | malabsorption of fats, e.g., gallbladder and | |
| | | | | | pancreas insufficiency. | |
| | | | | | *Serum: For scientific purpose | |
| | | | | | *Technical device:- | |
| Gamma-tocotrienol | 1 | 15-20 mg | — | — | *Questionnaire: Conditions with accompanying | |
| | | | | | malabsorption of fats, e.g., gallbladder | |
| | | | | | and pancreas insufficiency. | |
| | | | | | *Serum:- | |
| | | | | | *Technical device:- | |
| Delta-tocopherol | 1 | 20-40 mg | — | — | *Questionnaire: Conditions with accompanying | |
| | | | | | malabsorption of fats, e.g., gallbladder and | |
| | | | | | pancreas insufficiency. | |
| | | | | | *Serum:- | |
| | | | | | *Technical device:- | |
| Delta-tocotrienol | 1 | 2-5 mg | — | — | *Questionnaire: Conditions with accompanying | |
| | | | | | malabsorption of fats, e.g., gallbladder and | |
| | | | | | pancreas insufficiency. | |
| | | | | | *Serum:- | |
| | | | | | *Technical device:- | |
| Mixed Carotinoids (e.g., | 1 | 10 mg | | | | |
| Betatene™) | | | | | | |
| Lycopene (e.g., Lyco-O- | 1 | 2-4 mg | | | *Questionnaire: Conditions with accompanying | |
| Mato™) | | | | | malabsorption of fats, e.g., gallbladder | |
| | | | | | and pancreas insufficiency. | |
| | | | | | *Serum:- | |
| | | | | | *SNP: all SNPs which indicate prostate cancer | |
| | | | | | risk, old age, male, | |
| | | | | | *Technical device:- | |
| Zeaxanthin | 1 | 2.5-5 mg | | | Eye problems | |
| Lutein | 1 | 5-10 mg | | | Eye problems | |
| Vitamin A (Palmitate) | 1 | 21,500-5,000 IU | | | *Questionnaire: Conditions with accompanying | |
| | | | | | malabsorption of fats, e.g., gallbladder and | |
| | | | | | pancreas insufficiency. | |
| | | | | | *Serum: As Retinol, Serum 1 ml: 200-150 ng/ml | |
| | | | | | light protection | |
| | | | | | *SNP: . . . | |
| | | | | | *Technical device:- | |
| Cholecalciferiol (Vitamin D) | 1 | 200-400 IU | 400 IU | 2000 IU | *Questionnaire: Conditions with accompanying | |
| | | [10-20%] | | | malabsorption of fats, e.g., gallbladder | |
| | | | | | and pancreas insufficiency. Dark skin, | |
| | | | | | Osteoporosis, Postmenopausal state. Prostate | |
| | | | | | disease. Low sun exposure. | |
| | | | | | *Serum: As 25-OH Vit. D, Serum 1 ml: | |
| | | | | | 25-45 ng/ml, light protection, trapsort frozen | |
| | | | | | *SNP: Vitamin D-receptor | |
| | | | | | *Technical device: Osteoson®-Kollagenoson, | |
| | | | | | BIA, \hat{ } | |
| Ubiquinol (Coenzyme Q10) | 2 | 30-200 mg | | | | |
| Vitamin K1/K2 | 1− | 2-5 mg/0.5-1 mg | | | | |
| Oligomer (Proanthocyanides, | 2 | 25-50 mg | | | | |
| OPC (e.g., Activin™) | | | | | | |
| Ascorbypalmitat (lipid-soluble | 3 | 25-50 mg | | | | |
| Vitamin C) | | | | | | |
| Alpha Lipoic-Acid (ALA) | + | 100-400 mg | | | | |
| Selen (from yeast, or an | 1 | 50-200 μg* | 55 | 400 | *Questionnaire: Age, Smoking habits, Prostate | |
| organic as sodium | | | | | disease, Thyroid problems, | |
| selenite) | | [12.5-50%] | | | *Serum: Senum 1 ml: 70-100 μg/L, | |
| | | | | | *SNP: GSTM1, GSTP1, GSTT1, . . . | |
| | | | | | *Technical device: BMI | |
| Magnesium (e.g., magnesium- | 1 | 150-250 mg | 420 | 350 | *Questionnaire: Age, Gender, Osteoporosis, | |
| aspartate, magnesium oxide) | | | | | Posmenopausal state, Prostate disease, | |
| | | | | | Heart disease, Hypertension, Restless | |
| | | [42.86-71.4%] | | | legs . . . | |
| | | | | | *Serum: Serum 1 ml: 19-25 mg/L | |
| | | | | | *SNP” those who are relevant for heart health and | |
| | | | | | bone health | |
| | | | | | *Technical device: Osteoson®-Kollagenoson, | |
| | | | | | BIA, \hat{ } | |

*UNLESS INDICATED DIFFERENTLY

Of course the invention method may also be used in the preparation of cosmetic compositions. Typical ingredients for cosmetic compositions include the following.
Retinol (Vitamin A)
   it significantly reduces the depth of lines and wrinkles
   it increases the elasticity of the skin
   it actively protects against UV damage deep inside the skin
   dosage range: 0.005-0.2%
Sodium Ascorbyl Phosphate (Vitamin C)
   it is activated in the skin to free Vitamin C
   it inactivates harmful oxidants
   it stimulates collagen synthesis
   dosage range: 0.2-5%
Vitamin E acetate
   it deactivates free radicals in the skin
   dosage range: 0.5-5%
Panthenol (Provitamin B5) and Phytantriol
   Panthenol facilitates the skin's ability to retain moisture
   Phytantriol enhances the Panthenol effect for a fresher, smoother skin
   dosage range: 0.1-5%
Bisabolol (Sesquiterpene alcohol)
   it accelerates the healing process in skin (anti-inflammatory effect)
   dosage range: 0.01-1%

The above materials are provided as examples. Of course, many other cosmetically effective ingredients may be used in the invention process to prepare cosmetic compositions. Additional ingredients include but are not limited to gamma-linolenic acid (GLA), coenzyme Q10, vitamin F liposomes, hyaluronic acid as well as oils including jojoba oil and macadamia nut oil. Common cosmetic ingredients such as emulsifiers, stabilizers, thickeners and fragrances may be included.

The preparation of cosmetic products may be carried out in multi-step mixing operations. By using a multi-step mixing process it is possible to avoid any adverse reactivity or incompatibility with different ingredients in the cosmetic product.

The end user may complete a questionnaire to provide data regarding for example to the end user's physical, emotional, nutritive and personal perception characteristics. The questionnaire may be used to collect subjective and/or objective data. The questionnaire may inquire and obtain details regarding the end user's personal lifestyle, dietary habits, micronutrient consumption, prescription drug therapy, non-prescription drug therapy, and medical history. The questionnaire may include general identifying data and general dietary data combined with objective and subjective medical condition data. In the initial consultation with the client for determining the basic information before proceeding to prepare a test formulation, the end user may be requested to complete a computerized questionnaire. The questionnaire captures such data such as name, address, age and gender of the end user. Further captured data may include any allergies or reactivities the end user has and which may be known. Lifestyle choices may be recorded together with nutritive habits.

Objective medical condition data may include the end user's response to whether or not any one of, for example, the following medically diagnosed conditions have been diagnosed; anemia, cancer, endometriosis, GERD (gastrointestinal esophageal reflux disease), sores (canker, cold), IBS (irritable bowel syndrome), MS (multiple sclerosis), ulcers, arthritis, Candida, gall bladder, heart disease, hyperthyroid, kidney disease, psoriasis, UTI (urinary track infection), asthma, diabetes, fibromyalgia, heptatis, hypothyroid, lactose intolerance and rosacea. Subjective medical condition data may question the end user with regards to the end user's tendency towards infection, frequency of colds or infections, presence of allergies, ability to heal, tendency towards bruising and other information or data that may not necessarily be medically diagnosed and may be affected by the end user's subjective determination.

The questionnaire may further solicit data with regard to the condition of the end user's skin, teeth and nails, internal organs, bones and joints, head and physical experiences such as trauma, and attitude and energy level.

In another aspect, the questionnaire may inquire about the end user's emotional or psychological state. The questionnaire may inquire about, for example, whether or not the end user is moody or depressed; stressed out; nervous and unable to relax; listless and irritable; energy level; grouchiness; and tiredness. Family history data, exposure to chemicals or hypersensitivity conditions may also be included.

In addition to the questionnaire, the end user may be presented with a series of analytical tests to provide objective data for the database. Criteria such as hydration of the skin, skin surface lipid structure, water transportation through skin and the skin structure may be determined through commercially available analyzers such as those available from Courage+Khazaka Electronic GmbH.

Of course, analysis of body fluids may be undertaken to determine micronutrient levels such as those of vitamins, minerals and other materials of the end user. Such bodily fluids include saliva, hair, blood, buccal mucosa, etc. Such tests may be undertaken with commercially available diagnostic kits such as the Great Smokies Diagnostic Laboratory kits for anti-oxidant analysis or mineral analysis.

Analysis of tissue samples provided by the end user may be undertaken to detect single nucleotide polymorphisms and thereby determine if the end user has any predisposition to certain diseases or bodily conditions which may be affected by a product preparation.

These tests and questionnaires may be presented only one time at the initial preparation or may be repeated over time to help identify and/or optimize a particularized product preparation for the end user.

Objective data may be obtained through a number of analytical and measurement techniques. Body composition may be measured with a technique such as body-impedance analysis (BIA). BIA can make an assessment of an individual's body composition including lean body mass, fat mass, and body water content. Both the end user's prior history of BIA, when available, and present BIA may be included in the information provided as objective input. The end user's response after undergoing a nutritional regime and/or physical exercise may provide a way of correlating stresses and/or impacts upon the individual and the resulting effects demonstrated on objective measurement criteria of the individual's body.

Collagen structure may be measured with ultrasound such as an Osteoson-Collagenoson apparatus at high frequency (22 MHz). Skin parameters such as mechanical properties can be measured using, for example, a Reviscometer such as the RVM600 by Courage+Khazaka. Mechanical characteristics of collagen in skin may provide, for example, elastic and viscoelastic properties of human tissue such as collagen and elastin fibers.

Bone density is also a measurement which may provide objective inputs for the process. Disturbances in bone tissue or bone tissue mass may be measured with a Osteodin device which may determine, for example the amount of bone loss, the amount of bone loss expected for an individual matching the objective criteria, physiological age of bone, and an expected period of osteoporosis.

Genetic data may also be included in the objective input. Gene analysis including single nucleotide polymorphisms (SNP) which indicate that the individual has a small change in the gene compared to the population at large may be included with the objective inputs. SNPs may be correlated with higher risks for particular diseases.

An individual may submit to a gene analysis by providing a cheek tissue swab for example. The identification of any SNP may be entered in the objective database for the individual. SNPs may be correlated in the empirically derived database to conditions such as cardiovascular health; metabolism of vitamins; detoxification such as metabolism of xenobiotics and environmental toxins (GSH-S-transferases); antioxidative enzymes which for example may neutralize free radicals and thereby destruct molecules involved in aging and a variety of diseases such as SOD; bone health, for example osteoporosis risk and vitamin D-receptor, collagen-1-alpha,-SP₁-polymorphism); inflammation; insulin sensitivity such as risk of diabetes mellitus type II; hormone metabolism such as metabolic destruction of estrogens; metabolism of ingested compounds such as alcohol and nicotine.

Body fluids may provide further objective data on the end user's condition and may further enrich the database data (e.g., inputs) for the end user. Conventionally known and widely applied tests such as a urine test, blood test, protein-loss test, and/or glucose test may provide data for the objective inputs. Testing regarding 2-16 hydroxy-estrogen-ratio; N-t low peptides; deoxapyradiline (DPD) crosslinks; DNS-oxidation (8-OHdG); 8-hydroxy-desoxy-guanacine. Testing of saliva can provide DHEA and cortisol results together with the sex hormone status such as 17-beta-estradiol, progesterone, and testosterone. Testing on fecal matter may provide an indication regarding polyposis and colon carcinomas through M2-Pk; helicobacter pylori-antigen, fructose-sorbit and zylet maldigestion.

The objective inputs may be first stored in a database. The objective inputs may be obtained from a third party such as a doctor or nutritionist whose economic interest lies in providing the analytical data rather than providing a product for long-term continuing sales to the end user. The subjective inputs may be obtained by consultation with an individual at the point of sale or by questionnaire through a networked computer device.

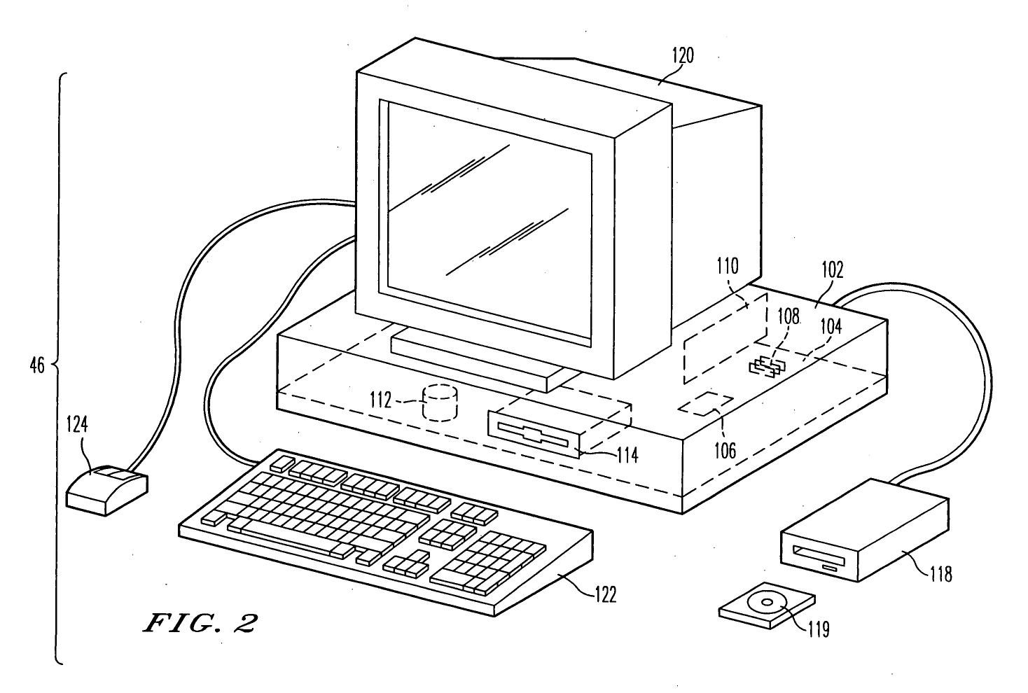

Balancing the objective and subjective inputs may be carried out with the use of a computer or microprocessor device. FIG. 2 is a schematic illustration of a computer system that may be used for carrying out a method for correlating subjective and objective inputs from an end user with an empirically derived database. In the computer system each of the empirically derived database, the objective inputs and subjective inputs may be stored permanently or transiently in one or more of a CPU or memory of the computer system. A computer 100 implements the method of the present invention, wherein the computer housing 102 houses a motherboard 104 which contains a CPU 106, memory 108 (e.g., DRAM, ROM, EPROM, EEPROM, SRAM, SDRAM, and Flash RAM), and other optional special purpose logic devices (e.g., ASICs) or configurable logic devices (e.g., GAL and reprogrammable FPGA). The computer 100 also includes plural input devices, (e.g., a keyboard 122 and mouse 124), and a display card 110 for controlling monitor 120. In addition, the computer system 100 further includes a floppy disk drive 114; other removable media devices (e.g., compact disc 119, tape, and removable magneto-optical media (not shown)); and a hard disk 112, or other fixed, high density media drives, connected using an appropriate device bus (e.g., a SCSI bus, an Enhanced IDE bus, or a Ultra DMA bus). Also connected to the same device bus or another device bus, the computer 100 may additionally include a compact disc reader 118, a compact disc reader/writer unit (not shown) or a compact disc jukebox (not shown). Although compact disc 119 is shown in a CD caddy, the compact disc 119 can be inserted directly into CD-ROM drives which do not require caddies. In addition, a printer (not shown) also provides printed listings of the results of the comparison including any subjective or objective input so that the end user may compare that data entered into the process with that data actually desired entered by the end user.

As stated above, the system includes at least one computer readable medium. Examples of computer readable media are compact discs 119, hard disks 112, floppy disks, tape, magneto-optical disks, PROMs (EPROM, EEPROM, Flash EPROM), DRAM, SRAM, SDRAM, etc. Stored on any one or on a combination of computer readable media, the present invention includes software for controlling both the hardware of the computer 100 and for enabling the computer 100 to interact with a human user. Such software may include, but is not limited to, device drivers, operating systems and user applications, such as development tools. Together, the computer readable media and the software thereon form a computer program product of the present invention for carrying out correlation and comparison between the inputted objective and subjective data with the empirically derived database. The computer code devices of the present invention can be any interpreted or executable code mechanism, including but not limited to scripts, interpreters, dynamic link libraries, Java classes, and complete executable programs.

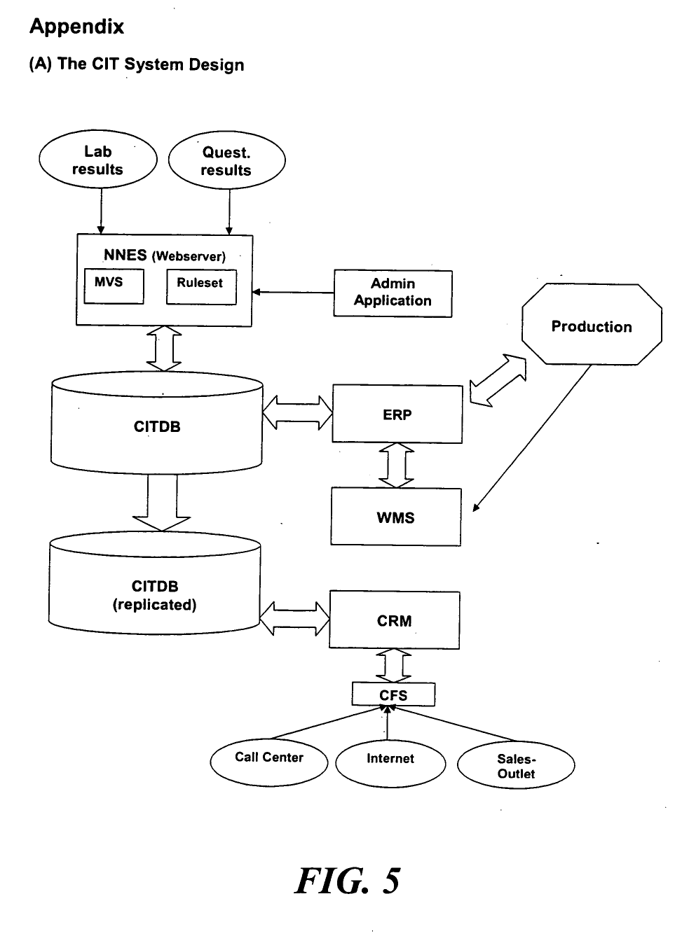

FIG. 5 shows a schematic depiction of a central IT system structure implementing a balance care (BC) product customization. The structure comprises a neural net expert system NNES which may be implemented as a web server. Data inputted to the NNES are objective data (such as lab results in the depiction of FIG. 5) and subjective data (such as questionnaire results in the depiction of FIG. 5). The NNES comprises a medical verification system MVS and a set of rules (RULESET) stored in the database. The set of rules are decision rules generated by the NNES, and a given decision rule describes which compound and what amount of this compound has to be added to the final custom prepared product according to the formulation derived as outlined above. An example for such a decision rule could be:
If customer “has a pancreas insufficiency” then
“add 50-100 mg of gamma-tocopherol to formula”

The decision rules as generated by the NNES might be categorized, e.g. by compound, questionnaire item, etc. in order to improve the system capacity and performance. Further, the set of rules can be set up to be editable during standard operations so that decision rules can be added, changed and/or deleted.

The medical verification system MVS of the NNES ensures the “medical integrity” of each of the above decision rules of the RULESET. The MVS in turn relies on a set of medical rules determining the correctness of a decision rule in respect to medical knowledge. A simple example for such a medical rule is:
If “dosage of gamma-tocopherol is >1,5 g per day”
then ALERT
An example for a more complex medical rule is:
If dosage of gamma-tocopherol is >750 mg per day and
customer has a liver problem then ALERT

If a decision rule violates a rule of the MVS, the system must alert all users and invalidate the formulation and—if already produced—the prepared product. The MVS thus provides for a layer of security which can also be very helpful when newly published medical knowledge is added to the verification system. It has to be emphasized that the administration of the MVS may only be performed by health care professionals. For this, the system comprises an Admin Application Module which offers a set of user rights restricting use of and access to various parts and modules of the overall system to authorized persons only, such as for example allowing access to the MVS to health care professionals only.

As already outlined above, the NNES processes input from two sources, namely objective data input and subjective data input, the result of the processing being stored in a database connected to the NNES, said database being called Central IT Database CITDB in the embodiment shown in FIG. 5. The data might be inputted into the NNES in a standardized format such as a markup language (e.g. XML), but any other appropriate data format can be used. As the input of the data can be done at a location remote to the NNES and/or the CITDB, the data may be transmitted via a Web Application Module using a secure connection (SSL).

The objective data and the subjective data inputted into the system may be structured into categories including the following three categories, each category triggering a different NNES action or a combination of NNES actions. A first category is immutable data, i.e. data of an end user which will not change over time such as for example gender, eye color, etc. A second category is data which does change over time, such as for example age of the end user, risk for certain diseases, etc., and a third category is data which can change over time (but which does not necessarily do so), such as for example an end user's weight, diseases etc. In the first category, the required NNES action may include reading and storing the data once. In the second category, the required NNES action is to adapt the formulation associated to the given customer automatically over time, and in the third category, the required action of the NNES may be to trigger collection of updated data periodically.

The NNES can then be designed to be able to adapt to changing end user parameters (second and third data categories) and thus changing ingredients and/or dosages in the formulation. This can be given for example in the following cases: the end user grows older (second data category) related to a possible automatic change of dosage; the medical condition of an end user changes (third category) related to a possible change of ingredient; new medical knowledge becomes available, seasonal influences (summer, winter, allergy season, influenza, etc.). An additional feature could be the offering of individual supplements, such as for example, special formulations for end users traveling to exotic or tropic countries with different environment conditions, diseases, infrastructure, etc.

The NNES workflow may include the following in any order, preferably in the order presented:
1. Collecting and aggregating input from two sources (objective and subjective data) and storing the raw data in the CITDB.
2. Using the set of decision rules to generate an end user profile (or retrieving/completing a profile for a returning end user).
3. Generating the formulation and storing it in the CITDB.
4. Transferring the formulation to the production system (Web-Service/XML).

The structure of the three categories can be supported by storing all (end user) profiles and formulations in a history file of the database of the NNES so that a NNES profile and formulation history is provided. Over a longer period of operation of the system, this history file forms the basis of a knowledge management system which can serve for studies based on profile changes, for recommendations for new end users based on the history of formulations of older end users.

As already described above, the system of balanced care product customization may comprise a dialogue with the end user (e.g., customer) as the end user actively creates and improves his or her “own” product. In order to assist this dialogue process, the system may further comprise a Customer Relationship Management (CRM) Module collecting input from several sources, such as for example from a call center, an internet application (website), a POS, etc. (as shown in the depiction of FIG. 5). The collection of information and data may be done via a Customer Feedback System CFS which may be part of the CRM module. The main task of the CFS consists in collecting, storing and structuring customer feedback from the various sources.

The purposes of the CRM module are manifold, and comprise, amongst others, building the foundation of an excellent customer service; enabling real-time personalization of the system's dialogue website; offering campaign management; offering an analytic platform on customer behavior (using for example OLAP and Data Mining); etc. These operations involve high volume access to the database CITDB so that it might be advantageous to replicate the CITDB (as indicated in FIG. 5) and connect the CRM module to the replication CITDB in order to avoid performance degradation on the production system. Of course, the CRM can also be outsourced to an application service provider that hosts the CRM solution for a fee.

Further and as shown in FIG. 5, the system may comprise an Enterprise Resource Planning (ERP) module that manages all orders and the inventory wherein basic order data is stored in the CITDB and transferred to the ERP module. The ERP module is the linking element between the CITDB and the production system. Particularly, the ERP module controls and triggers all production systems. The ERP module might be connected to a Warehouse Management System WMS that is responsible for controlling movement and storage of material within an operation and for processing associated transactions. Further, it is possible to provide for an automated returns management (or reverse logistics) as indicated by the arrow in FIG. 5 pointing from “Production” to the WMS, encompassing all of the processes associated with receiving a shipped product back into a warehouse facility or distribution center and dispositioning it for warranty repair, liquidation, return to customer, etc.

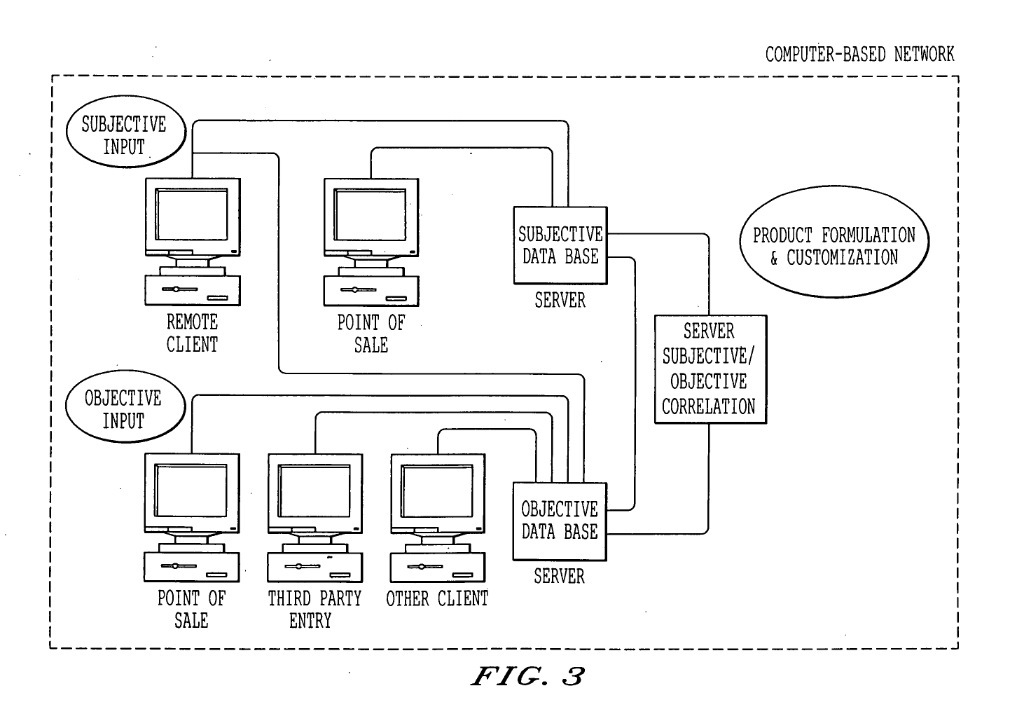

FIG. 3 shows embodiments of the invention process and their relation to a computer-based network. The computer-based network may include one or more remote clients which may be a computer located in the proximity of an end user. The remote client can be located for example, at the end user's domicile. Information and data inputted or processed by the remote client may be transferred to any number of the other nodes in the computer-based network. The information or data is transmitted electronically, for example, over the Internet from the remote client to a subjective database where it may be stored or it may be directly transferred for compiling and/or analysis. The remote client may be connected via a network to one or more points of sale directly or through another node such as a database present in the computer-based network.

Access to the computer-based network may include access of a third party which may enter information or data, such as objective data regarding an end user's physical characteristics or health analysis through the computer-based network so that it may be included in the analysis for product formulation, preparation and/or customization. The subjective input may be entered independently of the objective input or the two may be entered at the same time through one or more of a remote client, point of sale, third party, or other client. The nodes of the computer-based networks may be interconnected to a server. A server may be a computer or another node.

In FIG. 3 the subjective and objective databases are shown apart. The objective and subjective databases may be brought together physically or through internetworking wherein the subjective and objective databases may communicate with one another through a network connection directly or through one or more nodes. A server may undertake the analysis of the subjective/objective data for developing a formulation or preparation.

Any number of client nodes may be internetworked in the computer-based network. Clients for objective or subjective inputs may include home computers internetworked through the Internet or through other electronic communication pathways.

A number of storefront operations may share the same collection of objective and subjective input. Information such as the database may be stored on a central server to which new data is constantly added and the database thereby enriched and improved. The database is not limited to access by storefront or geographically bound individuals. Access over a network such as the internet through wireless devices is also possible. Remote access permits the effective utilization of a corps of traveling consultants to permit a wider distribution of the technical training necessary to carry out the invention method.

In one embodiment of the invention the subjective and objective inputs, which may exist as separate data files or together as a single database, are merged and/or compared with the empirically derived database. Comparison of the inputs with the empirically derived database allows identification of a series of variables for modification or sensitization through a customized product formulation. For example, the merged inputs and empirical database may be subjected to a mathematical algorithm which allows identification of specific variables such as, for example, micronutrient levels, which may be effected by the customized product which is eventually administered to the end user.

The algorithm, mathematical process or means of comparing the inputs with the empirically derived database may be derived through an iterative learning process whereby through repeated cycles using different input amounts and different input databases the sensitivity of certain variables to components of one or more customized product formulations are determined. An artificial neural network may be used to determine the sensitivity of any variable to different components that may be present in a customized product. By identifying the most sensitive variables and their related symptoms/effects, it may be possible to develop and prepare compositions which are responsive to both subjective and objective end user inputs and therefore provide the desired effect for the end user. It is preferred that the algorithm and artificial neural network are carried out on an automated basis with the aid of a computer. In one embodiment the sensitivity of a variable may be correlated in a linear manner to the administration of a component in a customized preparation and symptoms which manifest themselves to the end user. For example, an end user may provide objective data that indicates that the end user's skin elasticity is lower than what may normally be expected for an individual of that particular race, age and environmental influence. The end user may also input subjective information indicating that it is the desire of the individual to improve skin texture and/or skin elasticity. When the inputs are merged with the empirical database and a computerized algorithm is carried out, it may be determined that a certain nutrient, such as for example, vitamin A, is deficient or should otherwise be fortified in a skin cream for the end user. Through an iterative learning process or an artificial neural network the most beneficial dosage of the vitamin may be determined along with the form which may allow the most efficient administration.

Initially, the artificial neural network may be trained by input and guidance provided by a skilled practitioner. For example, a technician observing the skin of an individual who may benefit from improved viscoelastic properties of the skin may be able to direct the artificial neural network towards a solution wherein a known sensitizer of skin properties (e.g., vitamin A) may be modified in order to achieve an effect on the skin.

Artificial neural networks and the algorithms used therein are described in D. E. Rumelhart, G. E. Hinton, and R. J. Williams, “Learning Internal Representations by Error Propagation,” in D. E. Rumelhart, J. L. McClelland, eds. Parallel Distributed Processing: Explorations in the Microstructure of Cognition. Vol. 1, Cambridge, Mass.: MIT Press, pp. 318-362 (1986) (incorporated herein by reference).

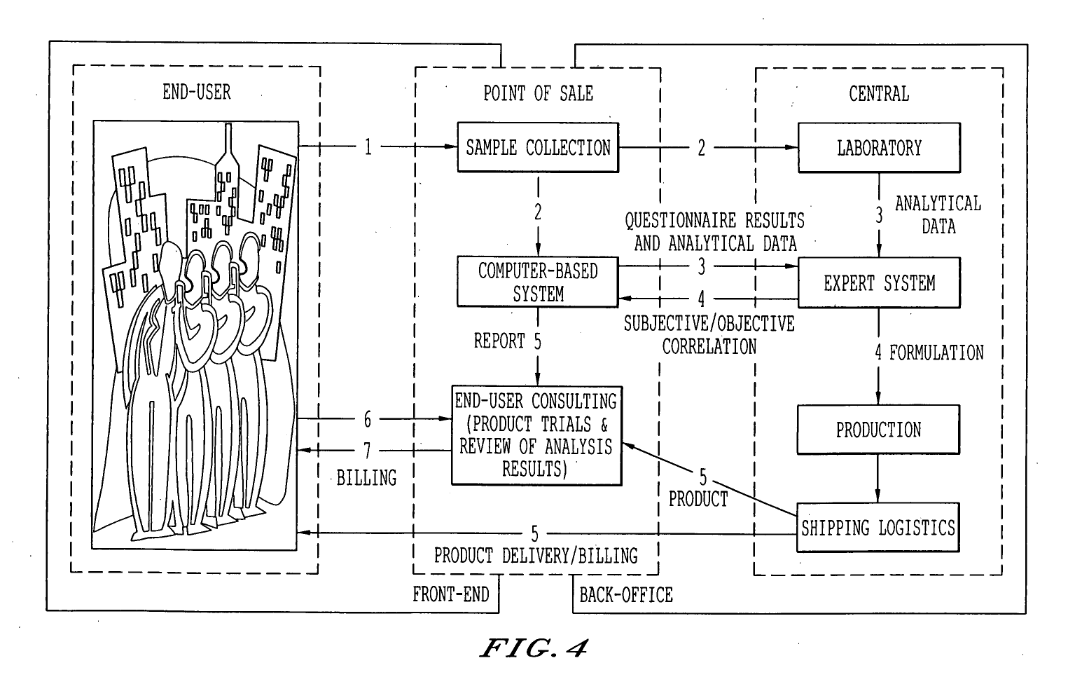

As shown in FIG. 4, the end user may be interviewed and tested and the resulting data may be entered into a computer-based system (2). This data may then be complied and/or included in a database and transferred to a central data technology receiver such as a server or other storage and/or processing unit (3). Through processing of this data, for example through an expert system, results and recommendations can be prepared for the end user (4). The results may include a product formulation for a specific end user and may be reported back to the end user (6) after first being communicated to a production facility for preparation (4). The product itself may then be transferred to the end user (5).

The method in this embodiment of the invention may therefore be broken into front-end and back-office functions. Front-end functions interface with the end user whereas the back-office functions are transparent to the user until the user is presented with a product for testing.

In addition to delivering the product the end user may be invited for a second consultation with the technician or sales representative. The second consultation may be used to fine-tune the formulation and get further data from the end user. In another embodiment, the second consultation with the end user may take place after the result of the correlation of the objective and the subjective data, and thus the derived formulation is available before final production and delivery of the customized product, enabling “fine tuning” of the final product and education of the end user. In this embodiment, a third consultation may take place upon delivery of the product.

An iterative learning process functions in a similar manner. By repeating a number of trials wherein objective and subjective criteria (e.g., inputs) are correlated with an empirical database and the scientifically derived relationships contained therein, it may be possible to develop a predictive understanding of the relationships between the inputs and the formulation of any customized product.

The interaction with the end user and its determination of a formulation or preparation may be carried out in cycles. Normally an end user may make an appointment or provide a sample or present himself for analysis at a storefront operation or be visited by a remotely working individual. After obtaining the objective and subjective data and cross-correlating the data from tissue samples or measurements on the end user an initial recommendation and product formulation is prepared. The product formulation is then discussed with the end user in a consulting meeting in a third step of the method. Consulting is preferably undertaken on a face-to-face basis. Consulting may be carried out, for example, in a doctor's office, a pharmacy, a cosmetic institute, or a department store and is preferably carried out in the storefront of the organization offering a balanced care customized formulation. As already mentioned above, consultation is carried out in every stage of the process by technically and scientifically trained staff.

In a preferred embodiment a feedback system is present. Feedback provides a way for the customer to constantly optimize the product to his or her changing needs while concurrently enriching the performance/product database. The submission of both objective and subjective inputs may therefore allow the end user to more quickly and more effectively identify and purchase a desired preparation.

Consulting provides an opportunity for feedback and potential optimization of the product formulation. A further step of the process may include sale of the product to the individual. Subsequent to sale a number of follow up steps may be taken for further consultation, optimization of the product formulation and other steps which may facilitate the feedback mechanism.

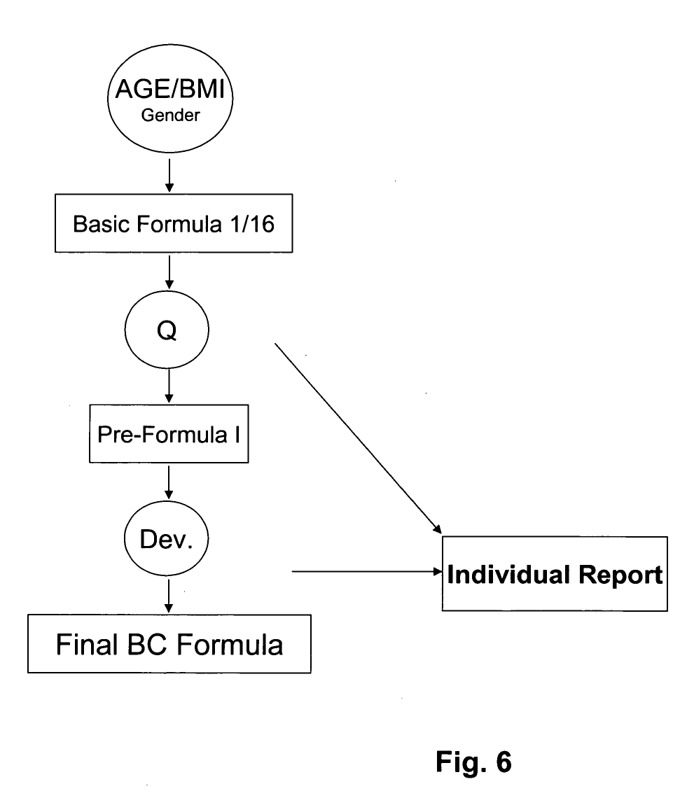

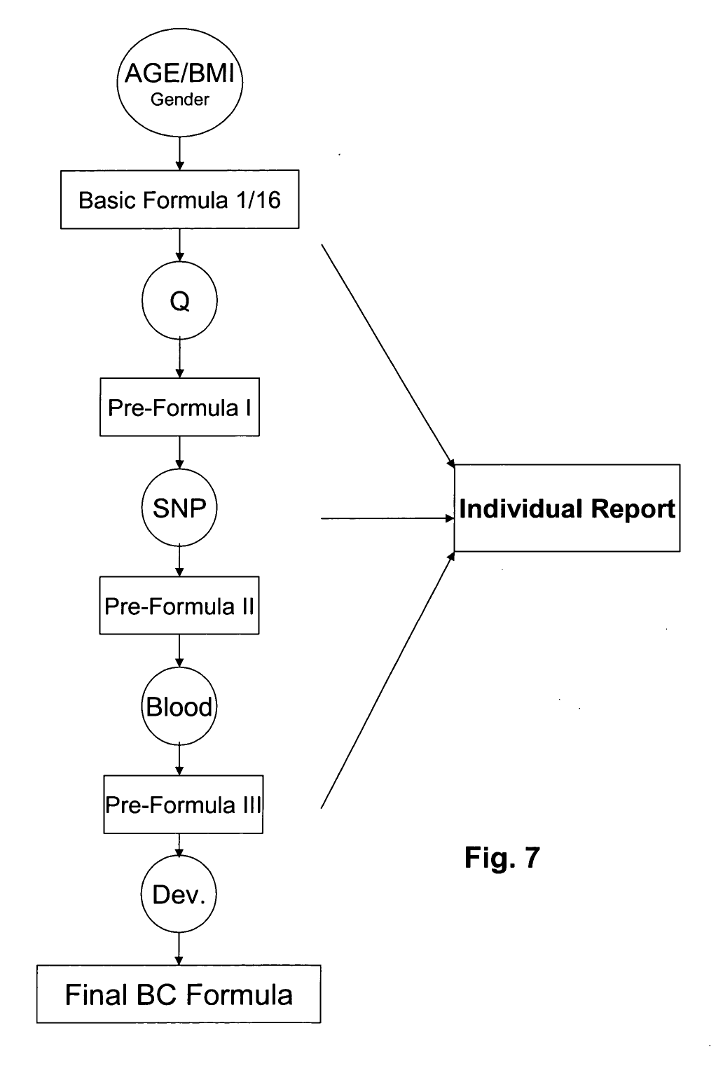

The process as described herein may be carried out in a stepwise manner when deriving the customized formula. Two possible embodiments are illustrated in the diagrams of FIGS. 6 and 7.

FIG. 6 shows a first embodiment in a schematic diagram. The first step of the process includes determining age and body mass index BMI of the end user, and preferably consists of determining age and body mass index BMI of the end user. Based on this finding, i.e. based on two parameters only, one out of a predetermined set of possible basic formulas is selected. In the embodiment shown, the set of basic formulas comprises 16 basic formulas one of which is selected as departing formula as this selected basic formula then subsequently has to be further amended in order to derive the final formula. This is done by evaluating the answers of the questionnaire as described above in detail which leads to a pre-formula (or intermediate formula) I. In a next step identified by “Dev.”, analysis results from devices (objective results) are included, such as for example body composition, bone density, skin analysis etc. and the final BC formula is derived. Parallely, an individual report on the basis of the analyses is drawn up by means of text modules linked to the various results of the analyses and their effects on the BC formula. The individual report may be the basis for the second and/or third consultation as described above.

FIG. 7 shows another possible embodiment of the process similarly to the depiction of FIG. 6. The process of FIG. 7 may be described as “enhanced” process with view to the process of FIG. 6 as it contains more steps of amending the basic formula into the final BC formula. Again and as in FIG. 6, the basic formula is chosen on the basis of two parameters, namely age and BMI. In this context, it has to be emphasized that the selection of the basic formula my be conducted on more or less parameters than two, and that it may be effected on the basis of different parameters than age and BMI, such as for example gender (with pregnancy as additional alternative), smoker/non-smoker, a given allergy etc. In the FIGS. 6 and 7, it is indicated that gender may be an additional criteria for selecting a basic formula. It might be possible to have a double set of 16 (or any other suitable number) of basic formulas for each gender, the gender selecting the set and age and BMI selecting one of the basic formulas contained in said set. The selecting may be carried out by any method, for example, selecting by the end user, selecting by a trained consultant, and selecting by computer. The selecting may include selecting the basic formula from a list of formulae, such as a list of end user formulae or reference formulae. The selecting from a list may be carried out by, for example, selecting from a list that is displayed as part of a menu and a list that is displayed as a graphical user interface.

As in the process of FIG. 6, the selected basic formula is amended into a first pre-formula I on the basis of the questionnaire evaluation. As a next step, the results from a gene test SNP are taken into consideration, resulting into a further amendment of the first pre-formula I into a second pre-formula II. Then, the results from a blood test are leading to further amendment into a third pre-formula III, and finally the third pre-formula III is amended into the final BC formula considering the results from device tests (cf. above FIG. 6). It has to be noted that the sequence of the steps illustrated in FIGS. 6 and 7 and described hereinbefore can vary without departing from the invention and without loss of quality in the result. It is therefore understood that the process of adapting a basic formula in an iterative manner in order to eventually obtain a final formula is not a linear process but can be designed modularly.

In one embodiment of the process, based on the answers to certain questions in the questionnaire or certain results of the tests, given ingredients corresponding scientifically to these answers or results are directly set to according values in the final formula. For example, if the end user answers the according question in the questionnaire that he or she is a smoker, the β-carotene dosage in the final formula is directly set to not exceed 10 mg and vitamin A is set to less than 2,500 IE, and the individual report will contain a text module inviting the end user to quit smoking and seek professional help to quit. If the end user states to be a vegan, vitamin B₁₂ is set 500 μg higher and Acetyl-Carnitine is set 500 mg higher, and the individual report will contain a text module informing about veganism and possible micronutrient deficiencies resulting therefrom. As a further example, if the end user states to consume soft drinks daily, chromium (Cr) is set 100 μg higher, and the individual report will contain a text module pointing out the long term risks of high carbohydrate intake (obesity, insulin resistance). If the end user states to drink more than e.g. three cups of green tea daily, there is no (direct) consequence on the final formula, but the individual report will contain a text module praising the end user and encouraging him to continue, outlining the multiple health benefits of green tea polyphenols. This is of course a limited choice of examples indicating the person skilled in the art how to set up a process according to the invention.

An according link list linking settings of the final formula as well as text modules for the individual report to results of the gene test may also be provided for in the database. For example, if the SNP gene test reveals MTHFR_C677T (Ala222Val) (Methylenetetrahydrofolate reductase), the following nutrigenomic intervention on the final formula may be the result:
1. Folate:=1000 μg+more
   Vitamin B₁₂:+800 kg
   Vitamin B₆:+50 mg
   Riboflavin B₂:+50 mg
2. Folate=1000 μg
   Vitamin B₁₂:+400 μg
   Vitamin B₆:+25 μg
   Riboflavin B₂:+50 mg
3. Folate:
   Vitamin B₁₂:
   Vitamin B₆:
   Riboflavin B₂:
for 1. the TT variant of the MTHFR_C677T-polymorphism, 2. the CT variant, or 3. the CC variant. The counsel report may then include the appropriate of the following text modules for these variants, respectively:

1. TT: Genetic testing has revealed the TT-variant of the MTHFR_C677T-polymorphism. It is known from published studies that this variant is associated with an approximately 55-% reduction of activity of the enzyme MTHFR. This leads to increased formation of homocysteine an amino-acid metabolite, which causes harm to vascular integrity, thereby increasing the risk of cardio-vascular diseases. Increased homocysteine levels have also been found in association with osteoporosis, Alzheimer's-dementia and non-Alzheimer's dementia. The metabolic control of homocysteine formation (and degradation) depends on the consistency of your enzymatic equipment (genotype) in relation to the presence of the B-vitamins, B₂, B₆, B₁₂ and folate. If, like in your case, the enzymatic activity of MTHFR is lower than in the CT, or CC-variants, more folate is needed to attain normal homocysteine levels. However, the other B-vitamins should also be present at certain levels. This has been taken into account for your BC-formula. The T-allele associated with reduced colon cancer and acute lymphocytic leukaemia risk. The protective may be due to the fact that leukaemia (as well as colorectal cancers) are derived from rapidly dividing tissues that have greater requirements for DNA precursors. However high alcohol intake and low folate consumption remove this protection. Overt alcohol consumption is generally not beneficial. In order to maintain the protective effect of your variant against colon cancer we recommend to restrict your alcohol intake.

2. CT: Genetic testing has revealed the CT-variant of the MTHFR_C677T-polymorphism. It is known from published studies that this variant is associated with an approximately 30-% reduction of activity of the enzyme MTHFR. This leads to increased formation of homocysteine an amino-acid metabolite which causes harm to vascular integrity, thereby increasing the risk of cardio-vascular diseases. Increased homocysteine levels have also been found in association with osteoporosis, Alzheimer's-dementia and non-Alzheimer's dementia. The metabolic control of homocysteine formation (and degradation) depends on the consistency of your enzymatic equipment (genotype) in relation to the presence of the B-vitamins, B₂, B₆, B₁₂ and folate. If, like in your case, the enzymatic activity is lower than in the CC-variant (but better as compared to the TT-variant), more folate is needed to attain normal homocysteine levels. However, the other B-vitamins should also be present at certain levels. This has been taken into account for your BC-formula.

3. CC: Genetic testing has revealed the CC-variant of the MTHFR_C677T-polymorphism. It is known from published studies that this variant, which is called the wild-type, is associated with a normally functioning enzyme MTHFR. In comparison to the other variants, CT, or TT, this leads to decreased formation of homocysteine, an amino-acid metabolite which causes harm to vascular integrity. Higher homocysteine levels can the risk of attracting cardio-vascular diseases and have also been found in association with osteoporosis, Alzheimer-dementia and non-Alzheimer dementia. The metabolic control of homocysteine formation (and degradation) depends on the consistency of your enzymatic equipment (genotype) in relation to the presence of the B-vitamins, B₂, B₆, B₁₂ and folate. If, like in your case, the enzymatic activity is “better” than in the CT, and CC-variants, low dietary intake of B-vitamins and high protein intake can still lead to increased homocysteine formation. Therefore, an appropriate amount of B-vitamins is needed. This has been taken into account for your BC-formula.

The process may be carried out on a point of sale (POS) system with or without guidance from a storefront operator or consultant. An end user may enter data directly at a point of sale system which allows entry of objective and subjective criteria. The end user may enter only subjective criteria or a combination of both subjective and objective criteria, all at once or in different sessions, depending on whether or not the objective criteria is provided directly to the end user or is first entered automatically into a database accessible through the POS. One advantage of a POS is that the end user may visit the POS several times without the need for a consultant or sales representative. The POS may also be used to rapidly identify the preferred formulation for the end user. An end user may continuously revisit the POS to update and provide feedback on already delivered products. This may initiate a feedback loop where data is rapidly updated in the subjective and objective inputs to quickly identify an optimum formulation for the end user.

The POS may consist of a computer having a CPU and memory which stores both the objective and subjective inputs as well as an empirically derived database. It is preferred that the POS is a client to a computer based system interconnected to a server. The server functions to store the subjective and objective data together with the empirically derived database. A remote location for the databases helps ensure security of any algorithms or comparative database methods which protect the customer formulation from piracy or vandalism.

The client may be directly connected to the server through a hard land line or by wireless communication for example.

The end user may input subjective data in the POS through a quick questionnaire that allows keyboard or mouse selection of certain answers in the questionnaire. In one embodiment of the invention the data (e.g., subjective and objective inputs) is entered through a mobile device such as wireless phone. In order to reduce the costs of the POS it is preferred that the calculations and algorithmic comparisons are undertaken on the server and that the client acts only as a point to display and enter data.

The POS may include a microprocessor and memory for transient comparison of the inputted data and the empirically derived database which may be present transiently in the memory of the POS or on a remote server. Therefore, the POS may directly calculate the desired formulation in one embodiment of the invention. The calculation is then transferred directly back to the server for storage on a separate database and transferred to a formulator for preparation of the end user's desired composition.

In one aspect of the invention the POS may be equipped with analytical equipment which allows objective data to be measured directly at the POS. Such analytical data may include color measurement equipment which allows a determination of, for example, skin color.

When a preparation is complete, a message may be directly transmitted to the end user if the end user has provided other identifying data such as a personal telephone number to a wireless device such as a wireless telephone, PDA or a pager.

In another aspect of the invention the end user is provided with a calendar indicating dates at which times a particular preparation may expire and is no longer usable and reminders to visit a consultant/physician. Reminders may be sent to obtain refills for preparation. The end user may also reorder materials directly through either the POS or through another device such as a telephone network or a wireless telephone so that new material is delivered directly to the end user without interruption of the treatment regimen.

Provisional applications 60/600,374 and 60/536,837 are each incorporated herein by reference in their entirety.

1. A computer-based method for formulating a custom product for an individual, comprising:
inputting objective data determined from the individual,
inputting subjective data provided by the individual, and
deriving a formulation for a customized product for the individual by correlating the objective and subjective inputs with empirically derived data.

2. The method according to claim 1, wherein the objective data is obtained from tissue samples or bodily fluids of the individual.

3. The method according to claim 1, wherein the objective data and the subjective data are stored in different databases.

4. The method according to claim 1, further comprising
obtaining product performance data from the individual to change the custom formulation.

5. The method according to claim 1, wherein at least one of the objective data and the subjective data is stored at a physical location remote from the location at which the data is inputted.

6. The method according to claim 1, wherein the subjective data is obtained from a questionnaire completed by the individual in the presence of a technically trained consultant.

7. The method according to claim 1, further comprising
reviewing the objective inputs with the individual to determine if a subjective input is changed.

8. The method according to claim 1, wherein a medical doctor is not present while the subjective data is obtained.

9. The method according to claim 1, wherein a medical doctor does not contact the individual.

10. The method according to claim 1, wherein the objective and subjective data is entered at a client terminal and stored at a remote server.

11. The method according to claim 1, wherein the empirically derived data comprises a correlation of nutrients, daily dosage data and effects on the human condition.

12. The method according to claim 1, wherein the correlation of the objective and subjective inputs with the empirically derived data is carried out with an artificial neural network.

13. The method according to claim 1, further comprising
subjecting the individual to a questionnaire to refine the subjective inputs before correlating with the empirically derived data.

14. The method according to claim 1, wherein the custom formulation is free of prescription medicines.

15. The method according to claim 1, wherein the objective data is obtained by carrying out an analytical measurement on one or more samples taken from the individual.

16. A computer program product including a computer readable medium and computer code embedded therein for controlling a computer processor to formulate a custom product for an individual, the computer code controlling the computer processor to at least perform the steps of:
inputting objective data determined from the individual,
inputting subjective data provided by the individual, and
deriving a formulation for a customized product for the individual by correlating the objective and subjective inputs with empirically derived data.

17. The computer program product according to claim 16, wherein the objective data is obtained from tissue samples or bodily fluids of the individual.

18. The computer program product according to claim 16, wherein the computer code further controls storing the objective data and the subjective data in different databases.

19. The computer program product according to claim 16, wherein at least one of the objective data or the subjective data is stored at a physical location remote from the location at which the data is inputted.

20. The computer program product according to claim 16, wherein the subjective data is obtained from a questionnaire completed by the individual in the presence of a technically trained consultant.

21. The computer program product according to claim 16, wherein the objective and subjective data is inputted at a client terminal and stored at a remote server.

22. The computer program product according to claim 16, wherein the formulation comprises a correlation of nutrients, daily dosage data and effects on the human condition.

23. The computer program product according to claim 16, wherein the correlation of the objective and subjective inputs with the empirically derived data is carried out with an artificial neural network.

24. The computer program product according to claim 16, wherein the objective data is obtained by carrying out an analytical measurement on one or more samples taken from the individual.

25. A system for formulating a custom product for an individual, comprising:
a first input section configured to input objective data determined from the individual,
a second input section configured to input subjective data provided by the individual, and
a calculation section configured to derive a formulation for a customized product for the individual by correlating the objective and subjective inputs with empirically derived data.

26. The system according to claim 25, wherein the objective data is obtained from tissue samples or bodily fluids of the individual.

27. The system according to claim 25, wherein the objective data and the subjective data are stored in different databases.

28. The system according to claim 25, further comprising a third input section configured to input product performance data from the individual to change the formulation.

29. The system according to claim 25, wherein at least one of the objective data and the subjective data is stored at a physical location remote from the first or second input sections.

30. The system according to claim 25, wherein the subjective data is determined from a questionnaire completed by the individual in the presence of a technically trained consultant.

31. The system according to claim 25, wherein the objective and subjective data is inputted at a client terminal and stored at a remote server.

32. The system according to claim 25, wherein the formulation comprises a correlation of nutrients, daily dosage data and effects on the human condition.

33. The system according to claim 25, wherein the correlation of the objective and subjective inputs with the empirically derived data is carried out with an artificial neural network.

34. The system according to claim 25, wherein the objective data is determined by carrying out an analytical measurement on one or more samples taken from the individual.

35. A computer-based method for formulating a custom product for an individual, comprising the following steps:
inputting objective data determined from the individual,
inputting subjective data provided by the individual,
selecting a basic formula as a basis for the custom product,
deriving a formulation for the custom product by adapting the basic formula based on the objective and subjective inputs according to at least one adaptation criterion.

36. The method according to claim 35, wherein the step of selecting the basic formula comprises selecting the basic formula from at least one list of available formulae.

37. The method according to claim 36, wherein the at least one list comprises a list displayed as part of at least one of a menu and a graphical user interface.

38. The method according to claim 35, wherein the adaptation criterion includes empirically derived data.

39. The method according to claim 35, wherein the selecting is carried out on the basis of at least one parameter contained in one or more of the objective or subjective inputs.

40. The method according to claim 35, wherein the selecting is carried out on the basis of at least two parameters contained in one or more of the objective or subjective inputs.

41. The method according to claim 38, wherein the at least two parameters comprise age and body mass index of the individual.

42. The method according to claim 35, wherein the objective data is obtained from at least one of tissue samples, bodily fluids or a gene test of the individual.

43. A computer program product including a computer readable medium and computer code embedded therein for controlling a computer processor to formulate a custom product for an individual, the computer code controlling the computer processor to at least perform the steps of:
inputting objective data determined from the individual,
inputting subjective data provided by the individual,
selecting a basic formula as a basis for the custom product, and deriving a formulation for the custom product by adapting the basic formula based on the objective and subjective inputs according to at least one adaptation criterion.

44. The computer program product according to claim 43, wherein the adaptation criterion includes empirically derived data.

45. The computer program product according to claim 43, wherein the selecting is carried out by controlling the computer processor on the basis of at least one parameter contained in one or more of the objective and subjective inputs.

46. The computer program product according to claim 43, wherein the selecting is carried out by controlling the computer processor on the basis of at least two parameters contained in one or more of the objective and subjective inputs.

47. The computer program product according to claim 46, wherein the at least two parameters comprise age and body mass index of the individual.

48. The computer program product according to claim 43, wherein the objective data is obtained from at least one of tissue samples, bodily fluids or a gene test of the individual.

49. A system for formulating a custom product for an individual, comprising:
a first input section configured to input objective data determined from the individual,
a second input section configured to input subjective data provided by the individual, and
a third input section configured to allow the individual, a different individual or a computer to select a basic formula as a basis for the custom product,
a first calculation section configured to derive a formulation for the custom product by adapting the basic formula based on the objective and subjective data.

50. The system according to claim 49, wherein the first calculation section includes a section for selecting the basic formula from at least one list of available formulae.

51. The system according to claim 50, wherein the list comprises a list displayed as part of at least one of a menu and a graphical user interface.

52. The system according to claim 49, wherein the adaptation criterion includes empirically derived data.

53. The system according to claim 49, wherein the objective data and subjective data are stored in different databases.

54. The system according to claim 49, wherein at least one of the objective data and the subjective data is stored at a physical location remote from the first, second or third input sections.

55. The system according to claim 49, wherein the subjective data is determined from a questionnaire completed by the individual in the presence of a technically trained consultant.

56. The system according to claim 49, wherein the objective data and subjective data is inputted at a client terminal and stored at a remote server.

57. The system according to claim 56, wherein the basic formula is inputted at a different client terminal and stored at a different remote server.

58. The system according to claim 49, wherein the formulation comprises a correlation of nutrients, daily dosage data, effects on the human condition and the basic formula.

59. The system according to claim 49, wherein the objective data is obtained from at least one of tissue samples, bodily fluids or a gene test of the individual.

60. A computer-based method for formulating a custom product for an individual, comprising the following steps:
inputting objective data determined from the individual,
inputting subjective data provided by the individual,
selecting a basic formula as a basis for the custom product on the basis of at least one parameter contained in the objective and subjective inputs,
adapting the selected basic formula according to a first subset of the objective and subjective inputs to obtain an intermediate formula,
adapting the intermediate formula according to a second subset of the objective and subjective inputs, and
obtaining a final formulation for the custom product.

61. The method according to claim 60, wherein the step of selecting the basic formula comprises selecting the basic formula from at least one list of available formulae.

62. The method according to claim 61, wherein the at least one list comprises a list displayed as part of at least one of a menu and a graphical user interface.

63. The method according to claim 60, wherein the adapting the intermediate formula is repeated for one or more additional subsets of the objective and subjective inputs before obtaining the final formulation.

64. The method according to claim 60, wherein the first subset comprises the subjective input.

65. The method according to claim 60, wherein the second subset comprises the objective input.

66. The method according to claim 65, wherein the objective input is obtained from at least one of tissue samples, body composition tests, bodily fluids or a gene test of the individual.

67. A computer program product including a computer readable medium and a computer code embedded therein for controlling a computer processor to formulate a custom product for an individual, the computer code controlling the computer processor to at least perform the steps of:
inputting objective data determined from the individual,
inputting subjective data provided by the individual,
selecting a basic formula as a basis for the custom product on the basis of at least one parameter contained in the objective and subjective inputs,
adapting the selected basic formula according to a first subset of the objective and subjective inputs to obtain an intermediate formula,
adapting the intermediate formula according to a second subset of the objective and subjective inputs, and
obtaining a final formulation for the custom product.

68. The computer program product according to claim 67, wherein the step of selecting the basic formula comprises selecting the basic formula from at least one list of available formulae.

69. The computer program product according to claim 68, wherein the at least one list comprises a list displayed as part of at least one of a menu and a graphical user interface.

70. The computer program product according to claim 67, wherein the adapting the intermediate formula is repeated for one or more additional subsets of the objective and subjective inputs before obtaining the final formulation.

71. The computer program product according to claim 67, wherein the first subset comprises the subjective input.

72. The computer program product according to claim 67, wherein the second subset comprises the objective input.

73. The computer program product according to claim 67, wherein the objective input is obtained from at least one of tissue samples, body composition tests, bodily fluids or a gene test of the individual.

74. A system for formulating a custom product for an individual, comprising:
a first input section configured to input objective data determined from the individual,
a second input section configured to input subjective data provided by the individual,
a first selection section configured to select a basic formula as a basis for the custom product on the basis of at least one parameter contained in the objective and subjective inputs,
a first calculation section configured to adapt the selected basic formula according to a first subset of the objective and subjective inputs to obtain an intermediate formula,
a second calculation section configured to adapt the intermediate formula according to a second subset of the objective and subjective inputs, and
a third calculation section configured to derive a final formulation for the custom product.

75. The system according to claim 75, wherein the objective data and subjective data are stored in different databases, wherein the third input section is separate from the first and second input sections,
wherein the first, second and third calculation sections are carried out in the same section.

76. The system according to claim 75, wherein the objective data and subjective data is stored at a physical location remote from the first, second or third input sections.

77. The system according to claim 75, wherein the objective data, subjective data and basic formula is inputted at a client terminal and stored at a remote server.

78. The system according to claim 75, wherein the adapting of the basic formula, the adapting of the intermediate formula, and the obtaining of the final formula is carried out with an artificial neural network.
